(19) United States
(12) Patent Application Publication
(10) Pub. No.: US 2005/0048555 A1
(43) Pub. Date: Mar. 3, 2005

(54) FRET imaging using an iterative estimation algorithm

(76) Inventors: Timothy J. Holmes, East Greenbush, NY (US); Yupeng Zhang, Rensselaer, NY (US); Yumin Yuan, Troy, NY (US)

Correspondence Address: HOFFMAN WARNICK & D'ALESSANDRO, LLC, 3 E-COMM SQUARE, ALBANY, NY, 12207, US

(21) Appl. No.: 10/920,619

(22) Filed: Aug. 18, 2004

Related U.S. Application Data
(60) Provisional application No. 60/497,589, filed on Aug. 25, 2003.

(51) Int. Cl. C12Q 1/68; G06K 9/00; C12M 1/34

(52) U.S. Cl. 435/6; 435/287.2; 382/128

(57) ABSTRACT

A method and method for processing fluorescence resonance energy transfer (FRET) image data. The method includes the steps of obtaining a set of FRET images and a set of calibration images for a biological sample; and generating a set of estimation images with an estimation algorithm that uses image data from the set of FRET images and the set of calibration images.

### REFERENCE TO PRIOR APPLICATION

The current application claims the benefit of co-pending U.S. Provisional Application No. 60/497,589, filed on Aug. 25, 2003, which is hereby incorporated herein by reference.

### BACKGROUND OF THE INVENTION

1. Technical Field

The present invention relates generally to microscope imaging processes, and more specifically relates to a system and method for generating fluorescence resonance energy transfer (FRET) images using an estimation technique, such as maximum likelihood estimation.

2. Related Art

Fluorescence resonance energy transfer (FRET) is a fluorescence microscope imaging process where energy is transferred nonradiatively from a fluorophore (the donor) in an excited state to a second fluorophore (the acceptor). It is a relatively new imaging method used in biological applications to study, monitor or measure protein-protein interactions. A FRET image is sensitive to whether or not two molecular components are bound to the same protein, because a FRET signal occurs only if the two protein components are within a few nanometers from each other, and therefore they are bound either to each other or to the same macromolecule.

Although relatively new to imaging, the general idea of FRET has been used with spectroscopy instrumentation for many years in biochemistry. The two protein components being studied are labeled with separate fluorophores. A quantum FRET event occurs when one of the labeled protein components (the donor) absorbs an excitation photon, and, rather than emitting a fluorescent photon, it transfers the energy to the other labeled protein component (the acceptor). A percentage of these FRET events will then be converted to a fluorescence emission from the acceptor. Such FRET events occur only when the two protein components are within a few nanometers from each other, and must therefore be bound to each other or to a common macromolecule. In a simplistic sense, FRET is detected then, and in turn the protein-protein interaction detected, by utilizing an excitation filter tuned to the donor dye and an emission filter tuned to the acceptor dye.

FRET is used for a broad range of applications. For example, FRET is used as the basis of an assay to detect the activity of caspase, which is a protease that facilitates apoptosis. It has been used to investigate interactions of cellular components such as membranes, intracellular ions and nucleic acids. It has been used to detect single nucleotide polymorphisms, which are variations in DNA sequences that cause diversity within species.

FRET may also be used to measure the distance between two proteins or between two fluorophores that are bound to the same protein. This measurement relies on an equation involving a FRET efficiency calculated from a set of images. Today, most biologists who use FRET are interested in knowing: (1) whether or not FRET is happening at all, and (2), if so, where it is happening (i.e., where in the cell).

The main limitations in existing FRET algorithms are due to two interrelated phenomena. (1) In order for FRET to occur, the emission spectrum of the donor fluorophore must overlap with the excitation spectrum of the acceptor fluorophore. Consequently the excitation and emission spectra of both dyes usually overlap and cross-talk and bleed-through are unavoidable. (2) As a result, the transfer equations that describe acquired images as functions of the desired FRET efficiency and other quantities do not have a closed-form solution. Accordingly, it is impossible to absolutely calculate a FRET image from a set of acquired images. Instead, existing algorithms rely on loosely justified approximations and assumptions to arrive at a solution. Arguably the two most prominent algorithms are those taught by Gordon, Herman et al., Quantitative Fluorescence Resonance Energy Transfer Measurements using Fluorescence Microscopy, Biophys. J., 74: 2702-2713, 1998, and the pFRET algorithm of Elangovan, Periasamy et al., Characterization of One-and Two-Photon Excitation Fluorescence Resonance Energy Transfer Microscopy, Methods 29: 58-73, 2003. The simplifying assumptions and approximations used by these algorithms limit the accuracy of their results.

There are other known techniques that do not rely on these simultaneous equations. One of these is the fluorescence lifetime imaging method (FLIM). The FLIM method requires special instrumentation. The fluorescence lifetime (i.e., the emission decay time of an excited dye, after the excitation is removed) shortens during FRET. Sophisticated pulsing lasers and gating electronics are used to detect this fluorescence lifetime and when it changes. Even though this approach has advantages, including that it circumvents the issues of cross-talk and bleed-through, it has a disadvantage that it requires expensive instrumentation.

Other known methods involve photobleaching. The donor photobleaching method relies upon the phenomenon that photobleaching is retarded by shorter lifetimes. The FRET efficiency is calculated from monitoring the percentage photobleaching at each pixel. Those pixels where FRET exists will exhibit less photobleaching. The acceptor photobleaching method works by, as the name implies, bleaching the acceptor. After the acceptor is bleached, the quenching of the donor disappears and a difference image with the uncorrected donor image will show the regions of FRET. The main limitation of both of these methods is that, by design, they are destructive to the cell so they work well with fixed samples and do not work well in live imaging.

Another solution uses a confocal microscope that records the spectrum of the fluorescence emissions. From this spectrum, contributions from the donor and acceptor are determined using mathematical deconvolution, and thereby eliminate the errors of cross-talk and bleed-through. The disadvantage of this approach is that it requires expensive hardware to record the spectrum at each pixel.

Accordingly, a need exists for a system and method for generating accurate FRET images that does not requires expensive hardware.

### SUMMARY OF THE INVENTION

The present invention addresses the above-mentioned problems, as well as others, by providing a system and method for processing FRET images using an estimation technique (such as maximum likelihood estimation) based on the optimization of a cost function to automatically compensate for bleed-through and cross-talk degradations. The technique uses a simple image acquisition scheme, works with, e.g., a standard widefield or confocal microscope, and requires no special hardware or software. Experimental results demonstrate the consistent accuracy in detecting a FRET signal and calculating FRET efficiency.

In a first aspect, the invention provides a fluorescence resonance energy transfer (FRET) processing system, comprising: an input system for receiving image data, wherein the image data includes a raw FRET image set; and a maximum likelihood estimation (MLE) image processing system that processes the image data and generates a set of MLE images, wherein the MLE image processing system includes an MLE algorithm and an iteration system.

In a second aspect, the invention provides a fluorescence resonance energy transfer (FRET) processing system, comprising: an input for receiving image data, wherein the image data includes a raw FRET image set; an image alignment system that automatically aligns pixels from the inputted raw FRET image set; and an image processing system that processes the image data and generates a corrected donor concentration image, a corrected acceptor concentration image and a corrected FRET image.

In a third aspect, the invention provides a method for processing FRET image data, comprising: providing a raw FRET image set for a biological sample; and generating a set of maximum likelihood estimation (MLE) images with an MLE algorithm that uses raw FRET image set.

In a fourth aspect, the invention provides an image processing system, comprising: an image collection system for collecting image data including a set of fluorescence resonance energy transfer (FRET) images; and a FRET processing system for processing the image data and generating a set of maximum likelihood estimation (MLE) images using an MLE algorithm.

### BRIEF DESCRIPTION OF THE DRAWINGS

These and other features of this invention will be more readily understood from the following detailed description of the various aspects of the invention taken in conjunction with the accompanying drawings in which:

FIG. 1 depicts a FRET processing system in accordance with the present invention.

FIG. 2 depicts an image collection system in accordance with the present invention.

FIG. 3 depicts a schematic diagram of the principles of the FRET process in accordance with the present invention.

FIG. 4 depicts experimental results using the FRET processing system in accordance with the present invention.

FIG. 5 depicts additional experimental results using simulated images and the FRET processing system in accordance with the present invention.

FIG. 6 depicts additional experimental results using positive control images and the FRET processing system in accordance with the present invention.

FIG. 7 depicts additional experimental results using negative control images and the FRET processing system in accordance with the present invention.

### DETAILED DESCRIPTION OF THE INVENTION

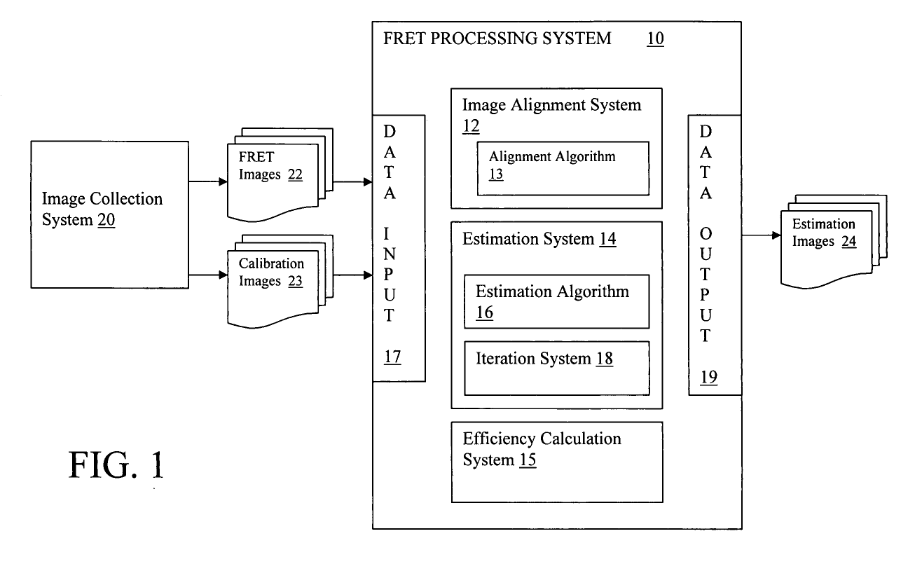

Referring now to the drawings, FIG. 1 depicts a FRET processing system 10 for processing images 22 acquired from an image collection system 20 and generating a set of estimation images 24. Image collection system 20 may comprise, e.g., a fluorescence microscope and camera for collecting image data, as well as a computer system and/or database or memory for storing acquired image data. FRET processing system 10 includes: (1) a data input 17 for receiving image data; (2) a data output 19 for outputting estimation images 24; (3) an image alignment system 12 for aligning image data from acquired images 22 and/or 23; (4) an estimation system 14 that estimates the corrected FRET image, as well as donor and acceptor concentrations; and (5) an efficiency calculation system for calculating FRET efficiencies.

While present invention is generally described with reference to imaging biological samples, it should be recognized that the invention could apply to any field, e.g., chemistry, polymers, biophysics, etc.

FRET processing system 10 may be implemented on any type of computer system, either as one or more software program products or as a combination of software and hardware. Moreover, FRET processing system 10 may be integrated with image collection system 20, or exist as a separate stand-alone system. In one exemplary embodiment, image alignment system 12 utilizes a spatial cross correlation process 13, described in further detail below. Estimation system 14 comprises an estimation algorithm 16 and iteration system 18 described below. In addition, as also described below, the acquired (or observed) images generally comprise nine images, including six images in the calibration image set 23 and three images within the raw FRET image set 22.

Estimation algorithm 16 may utilize any type of estimation technique that is based on the optimization of a cost function. In the exemplary embodiments described herein, estimation algorithm 16 is implemented and described using a maximum likelihood estimation (MLE) technique. However, it should be understood that other estimation techniques may be utilized, including maximum a posteriori (also called Bayesian); least squares; minimum mean-square-error; maximum entropy; maximum cross-entropy; MLE with penalties; any cost function with a mathematical constraint on the solution, sub-examples of constraints are, e.g., a smoothness constraint or entropy constraint; any cost function that includes a penalty term, etc.

In addition, iteration system 18 is described below using a “steepest ascent” technique to maximize the cost function. However, it should be understood that any type of iterative approach may be utilized. Other examples include: an expectation-maximization algorithm; conjugate gradient; simulated annealing; linear programming, etc. Moreover, it is also possible to utilize a “design-based” iterative approach that is not tied to the cost function.

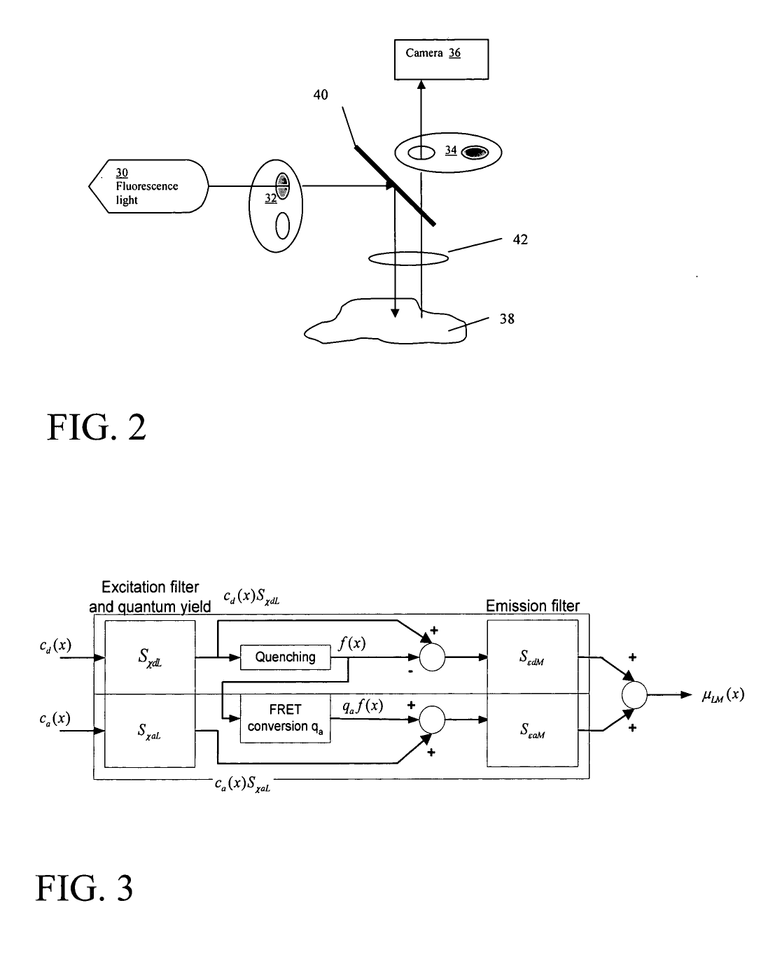

A simplified example of a FRET image collection system 20 is shown and described with reference to FIG. 2. The FRET image collection system may be implemented using any type of fluorescence acquisition device, such as a standard widefield or confocal microscope. Other types of acquisition devices may be a multiphoton microscope, a spinning disk confocal microscope. These devices may include those for imaging components of the eye, such as the posterior pole, retina, fundus or the cornea, presuming safety standards are met, including an ophthalmoscope, a fundus camera, digital fundus camera, a slit lamp camera or a scanning laser ophthalmoscope. In the example depicted in FIG. 2, a fluorescence light source 30 generates a light beam, which is passed through an excitation optical filter 32 (in this case set to the excitation wavelength of the donor). The light beam is then directed through a lens 42 to a sample 38 injected with one or more fluorophores. The light beam strikes the sample 38 with an excitation wavelength. This excitation light causes the dye to fluoresce either through direct fluorescent excitation or through the FRET process. The fluorescent light exits the sample with an emission wavelength. The exiting light beam then travels through an emission optical filter 34 (in this case set to the wavelength of the acceptor) and into a camera 36 where the image is collected.

FRET Process

The principles of the FRET process are shown schematically in FIG. 3, and the notations used in the method are shown in Table 1.

TABLE 1

| Notation | Definitions |
|---|---|
| x | 2D or 3D spatial variable |
| c_{d}(x) | Donor concentration in the biological sample. |
| cₐ(x) | Acceptor concentration in the biological sample. |
| Sₓ^{oL} | Excitation filter transfer efficiency with the dye o present and the filter tuned to dye L. The |
| | letter subscript o will indicate either d for donor or a for acceptor. The letter subscript L will |
| | indicate either D for donor or A for acceptor. |
| f(x) | Concentration of donor dye that is quenching due to FRET events. |
| F(x) | Normalized concentration of donor dye that is quenching due to FRET events. It is |
| | normalized to represents a part of the donor concentration. |
| qₐ | Quantum yield of fundamental radiative emission (fluorescence emission) of the acceptor. |
| | (R. M. Clegg, Fluorescence Resonance Energy Transfer, Ch. 7 in Fluorescence Imaging |
| | Spectroscopy and Microscopy, X. Wang and B. Herman, Ed's., Wiley, 1996.) This is the |
| | probability that an excited acceptor molecule will convert its excited energy state into an |
| | emission photon. |
| S_{εoM} | Emission filter transfer efficiency with the dye o present and the filter tuned to dye M. The |
| | letter subscript o will indicate either d for donor or a for acceptor. The letter subscript M will |
| | indicate either D for donor or A for acceptor. This transfer efficiency accounts for several |
| | physical factors, including the probability that the photon emits in a direction suitable to be |
| | directed to the camera, the probability that it is not absorbed or scattered by optical |
| | components along its way to the camera, the camera's sensitivity to its wavelength and other |
| | factors of light detection sensitivity. |
| μ_{LM}(x) | Image (noise free) that is collected by the camera when the biological sample is labeled with |
| | both donor and acceptor, the excitation filter is tuned to L and the emission filter is tuned to |
| | M, where L and M individually indicate either donor D or acceptor A. This notation is under |
| | the ideal assumption that the image is noise free. The image contains no quantum photon |
| | noise (the main contribution) or other noise, such as thermal noise from camera/photodector |
| | electronics, and readout noise which is a known problem to occur with CCD cameras.. |
| n_{LM}(x) | Image (with noise) that is collected by the camera when the biological sample is labeled with |
| | both donor and acceptor, the excitation filter is tuned to L and the emission filter is tuned to |
| | M, where L and M individually indicate either donor D or acceptor A. This image contains |
| | noise due to quantum photons. Although not considered in this simplified model, this term |
| | may also include other noise sources, such as those mentioned above. |
| μ_{oLM} | Calibration image (noise free) that is collected by the camera, where the letter subscript o |
| | indicates donor or accepter calibration sample, the letter subscript L indicates the dye to |
| | which the excitation filter is tuned, and the letter subscript M indicates the dye to which the |
| | emission filter is tuned. |
| M_{oLM} | Summation of the image taken with a calibration sample, where the letter subscript o |
| | indicates donor or accepter calibration sample, the letter subscript L indicates the dye to |
| | which the excitation filter is tuned, and the letter subscript M indicates the dye to which the |
| | emission filter is tuned. |

Note that while this embodiment is described with respect to inputting nine images, the invention could be implemented with more or fewer images. In one possible embodiment, a complete spectrum at each pixel could be utilized. In another embodiment, fewer than nine images could be utilized, as the minimization of the cost function does not depend on having all of the image data. Moreover, the filters utilized to generate the images do not have to be specifically tuned to the donor or acceptor, but rather could be tuned in any manner. For example, instead of being tuned to the donor, one of the filters may be tuned to a wavelength that is near, but not equal to, the characteristic wavelength of the donor or it may be tuned to a wavelength that is between the wavelengths of the donor and acceptor.

Inside of the specimen 38, there is a donor dye concentration c_{d}(x) and an acceptor dye concentration cₐ(x), where x is a 2D or 3D spatial coordinate. The model of the process is divided into two channels, a donor channel, which maps the signals flowing from the donor concentration, and an acceptor channel, which maps the signal flowing from the acceptor concentration. The excitation filter is tuned to either the acceptor or donor and has a transfer efficiency of S_{χoL}, where the subscript L denotes which dye the filter is tuned to and the subscript o denotes the dye that is present (see Table 1). The excited concentrations of the donor and acceptor, respectively, are c_{d}(x)S_{χdL} and c_{d}(x)S_{χaL}. The concentration of donor that fluoresces is reduced by f(x). The term f(x) is the concentration of excited donor molecules that become quenched due to FRET. The effective excited concentration equal to
c_{d}(x)S_{χdL}−f(x)=(c_{d}(x)−F(x))S_{χdL} (1)
where
F(x)=f(x)/S_{χdL}. (2)

With each quenching event, instead of the energy being converted to an emission photon, it is transferred to a nearby acceptor molecule. A fraction qₐ of these FRET transfers will be converted into an acceptor photon emission. The term qₐ is the quantum yield of fundamental radiative emission of the acceptor. Therefore, the number of photon emissions coming from the acceptor is increased by qₐF(x)S_{χdL}. The emission filter is tuned to either the acceptor or donor and has a transfer efficiency of S_{εoM}, where the subscript M denotes the dye to which the filter is tuned and the subscript o denotes the dye that is present. This transfer efficiency S_{εoM} accounts for several physical factors, including the probability that the photon emits in a direction suitable to be detected by the camera, the probability that it is not absorbed or scattered by optical components along its way to the camera, the camera's sensitivity to its wavelength and other factors of light detection sensitivity.

The number of photons that are, in effect, counted by the camera at position x then is equal to the summation of photons exiting the two channels and is expressed as:
μ_{LM}(x)=(c_{d}(x)−F(x))S_{χdL}S_{εdm}(cₐ(x)S_{χaL}+qₐF(x)S_{χdL})S_{εaM} (3)
This equation represents the ideal image that is collected when no noise is present. In the realistic case, there is noise due mainly to quantum photons. To account for this noise, the number of photons n_{LM}(x) is considered to be a random variable that is Poisson distributed, having a mean parameter μ_{LM}(x) equal to Eq. 3. Eq. 3, thereby, represents the statistical expectation of the number of photons. Although not described in detail here, other noise sources may be considered and modeled. Thermal noise due to electronics in the camera is typically modeled as a Gaussian process, and there is readout noise typically associated with the frame readout schemes of the CCD cameras. An perfectly accurate formulation would combine these noise models within the probalistic modeling of n_{LM}(x). The Poisson process of quantum photon noise is considered here, only, because the quantum photons are the primary source of noise and because for practical purposes the model is being kept simple and tractable.

As noted above, three images are collected for the raw FRET image set 22 are taken using the same biological sample, where both donor and acceptor concentrations are present, resulting in three simultaneous equations that adhere to Eq. 3. This triplet is referred to as the raw FRET image set: (1) n_{DD}(x) with the excitation and emission filters both tuned to the donor; (2) n_{DA}(x) with the excitation and emission filters tuned to the donor and acceptor, respectively; (3) n_{AA}(x) with the excitation and emission filters both tuned to the acceptor.

The aim is to solve for the FRET signal F(x). All of the terms on the right hand side of Eq. 3 are unknown. Some of these terms are determined by using calibration images 23. Six calibration images 23 are collected, three of which are collected with a donor-only calibration sample and having the same three filter combinations as specified in the above paragraph, and three of which are collected with an acceptor-only calibration sample and, again, having the same three filter combinations as specified in the above paragraph. The statistical expectation of the calibration image 23 is denoted as μ_{oLM}(x), where the first subscript indicates donor or accepter calibration sample, the second subscript indicates the dye to which the excitation filter is tuned, and the third subscript indicates the dye to which the emission filter is tuned. According to the system model shown in FIG. 3, this quantity is expressed as:
μ_{oLM}(x)=cₒ(x)S_{χoL}S_{εoM} (4)
The summations M^{oLM} of each of these calibration images is taken, according to

$${M}_{oLM} = {\sum }_{x} {μ}_{oLM} ( x ) , \qquad ( 5 )$$

eliminate noise effects. In other words, the value of the image intensity is summed for all pixels. Eq. 4 represents, in principle, 8 different expressions, since each of the 3 subscripts o, L and M takes on 2 possible values. In reality it represents 6 different expressions because the situation where the excitation filter is tuned to the acceptor (L=A) while the emission filter is tuned to the donor (M=D) is never used. By substituting Eq. 4 into Eq. 5 for all 6 cases (o=D, L=D, M=D; o=D, L=A, M=A; o=D, L=D, M=A; o=A, L=D, M=D; o=A, L=A, M=A; o=A, L=D, M=A), and then taking the ratios M_{dAA}/M_{dDA}, M_{aDA}/M_{aAA}, M_{dDA}/M_{dDD} and M_{nDD}/M_{aDA} we are able to express some of the efficiencies in terms of another. Thereby, the cross-talk coefficients S_{χdA}, S_{χaD}, S_{εaD} and S_{εdA} are expressed as:

$$\begin{matrix}{S}_{χ dA} = \frac{{M}_{dAA}}{{M}_{dDA}} {S}_{χ dD} & ( 6 ) \\ {S}_{χ aD} = \frac{{M}_{aDA}}{{M}_{aAA}} {S}_{χ aA} & ( 7 ) \\ {S}_{ɛ dA} = \frac{{M}_{dDA}}{{M}_{dDD}} {S}_{ɛ dD} & ( 8 ) \\ {S}_{ɛ aD} = \frac{{M}_{aDD}}{{M}_{aDA}} {S}_{ɛ aA} . & ( 9 )\end{matrix}$$

Eq. 6 to Eq. 9 may be substituted into the three equations of the raw FRET image set (Eq. 3), resulting in the following three expressions:

$$\begin{matrix}{μ}_{DD} ( x ) = [ {c}_{d} ( x ) - F ( x ) ] {S}_{χ dD} {S}_{ɛ dD} + {c}_{a} ( x ) \frac{{M}_{aDD}}{{M}_{aAA}} {S}_{χ aA} {S}_{ɛ aA} + F ( x ) \frac{{M}_{aDD}}{{M}_{aDA}} {q}_{a } {S}_{χ dD} {S}_{ɛ aA} & ( 10 ) \\ {μ}_{DA} ( x ) = [ {c}_{d} ( x ) - F ( x ) ] \frac{{M}_{dDA}}{{M}_{dDD}} {S}_{χ dD} {S}_{ɛ dD} + {c}_{a} ( x ) \frac{{M}_{aDA}}{{M}_{aAA}} {S}_{χ aA} {S}_{ɛ aA} + F ( x ) {q}_{a } {S}_{χ dD} {S}_{ɛ aA} & ( 11 ) \\ {μ}_{AA} ( x ) = [ {c}_{d} ( x ) - F ( x ) ] \frac{{M}_{dAA}}{{M}_{dDD}} {S}_{χ dD} {S}_{ɛ dD} + {c}_{a} ( x ) {S}_{χ aA} {S}_{ɛ aA} + F ( x ) \frac{{M}_{dAA}}{{M}_{dDA}} {q}_{a } {S}_{χ dD} {S}_{ɛ aA} & ( 12 )\end{matrix}$$

Ultimately, it is the solution of F(x) and c_{d}(x) that are desired, because F(x) represents the FRET signal and c_{d}(x) is used to calculate the FRET efficiency according to the ration F(x)/c_{d}(x). In order to affect this solution, all of the unknown parameters need to be solved. At this juncture, all of the terms on the right-hand-side of the equations, except for the M's, are unknown.

MLE Solution

Because there are more unknowns than there are equations, a closed form solution is not possible. The present invention utilizes a maximum likelihood estimation (MLE) system 14 that provides an iterative MLE solution, which is robust against adversities such as quantum photon noise. The criterion for the MLE solution is to maximize the log-likelihood expression. This log-likelihood is expressed by an MLE algorithm 16 as:

$$l = log \{ {\prod }_{x}^{} [ P ( {μ}_{DD} ( x ) ; {n}_{DD} ( x ) ) ] [ P ( {μ}_{AA} ( x ) ; {n}_{AA} ( x ) ) ] [ P ( {μ}_{DA} ( x ) ; {n}_{DA} ( x ) ) ] \} \qquad ( 13 )$$

where P(•) represents the Poisson probability distribution, and its first and second arguments are its mean parameter and random variable, respectively. It can be expressed as:

$$P ( {μ}_{LM} ( x ) ; {n}_{LM} ( x ) ) = \frac{{{μ}_{LM} ( x )}^{{n}_{LM} ( x )}}{{n}_{LM} ( x ) !} exp ( - {μ}_{LM} ( x ) ) \qquad ( 14 )$$

The formation is simplified by combining some of the terms into single quantities according to:
S_{DD}=S_{χdD}S_{εdD} (15)
S_{AA}=S_{χaA}S_{εaA} (16)
S_{DA}=qₐS_{dDs}S_{εaA} (17)
The set of unknown parameters c_{d}(x), cₐ(x), F(x), S_{DD}, S_{AA} and S_{DA} make up the elements of a vector V. The elements of the vector are the values of c_{d}(x), cₐ(x) and F(x) at every pixel location x, and the terms S_{DD}, S_{AA} and S_{DA}.

Note that while the current embodiment is described with reference to a Poisson probability distribution, other distributions such as Gaussian, binomial, etc., could be utilized.

Iteration system 18 provides a scheme for maximizing Eq. 13. Initially, a first guess of each parameter must be set in order to seed the iterations. Logical first guesses are straightforwardly chosen. For example, the first guess of S_{DD}, S_{AA} and S_{DA} may be set to be 1.0 since they represent filter transfer efficiencies, and the first guess of c_{d}(x) may be set to be equal to n_{dd}(x), since this is a crude representation of the donor. The iterative estimate \overline{\hat{v}} of the vector \overline{v} may be carried out, e.g., using a steepest-ascent scheme, which is described by the following formula:

$$\hat{\overline{v}} ← \hat{\overline{v}} + αΔ \hat{\overline{v}} \qquad ( 17 )$$

where α is an overrelaxation parameter, described below. Each element Δ\hat{v}ᵢ in this steepest ascent vector is determined iteratively by executing the formula:

$$Δ \hat{{v}_{i}} = \frac{\partial l}{\partial \hat{{v}_{i}}} \qquad ( 18 )$$

where \hat{v}ᵢ is the i-th element in \overline{\hat{v}}.

The vector

$$Δ \hat{\overline{v}}$$

points in the direction of steepest ascent and the overrelaxation parameter α is solved numerically to affect the point that maximizes l along that direction.

Using Eq. 13, and the methods described above, values for each pixel x are obtained for the outputted MLE images 24. The most useful MLE images include: (1) the corrected donor concentration c_{d}(x), where the \hat{ } overscript means “estimate of” and implies the MLE of c_{d}(x); (2) the corrected acceptor concentration Ĉₐ(x); and (3) the corrected FRET image \hat{F}(x). Any known computer program language can be utilized to implement the MLE algorithm and related calculations.

A variation on the steepest ascent theme involves separating the parameters into 2 groups. By doing so, faster convergence of the iterations may be affected. An example selection of group 1 is the 3 parameters of Eq's. 15-17 and having these 3 parameters make up the the 3 elements of vector \overline{v}_{group 1} while the 3 images represented by cₐ(x), c_{b}(x) and F(x) make up the elements of \overline{v}_{group 2.} Instead of Eq's. 17 and 18 being executed exactly as shown, a variation is executed that is described by repeating Eq's. 17 and 18 with group 1 and group 2 subscripts on the \overline{v} terms and by executing those two expressions in order.

Other Models and Approaches

There are many other models besides the model illustrated in FIG. 3 that may be derived and utilized. For example, refinements to the model shown will result in a more accurately emulation of the true physical situation. Only one type of quantum efficiency qₐ is considered in the model shown, and there are others which could be added. After an acceptor takes on a high energy state, for example, it may give off its energy by nonradiatively exciting another nearby acceptor fluorophore. There are many second-order effects, like this, which are not described here and which could be modeled. Another possible refinement is one that considers the effects of variations in photodetector quantum efficiency of a CCD camera and of background levels seen in the photodetector elements of the CCD camera.

Besides using a cost function, a designer may take an intuitive approach to specifying the iterations. Examples of intuitively derived iterative algorithms, that utilize a forward model in other applications are the Algebraic Reconstruction Technique (ART) used in computed tomography (R. Gordon, “A tutorial on ART (algebraic reconstruction techniques),” IEEE Trans. Nucl. Sci., NS-21, 471-481, 1970), and the Jansson-van Cittert algorithm used in light microscopic image deconvolution (D. A. Agard, Y. Hiraoka, J. W. Sedat, “Three-dimensional microscopy: image processing for high resolution subcellular imaging,” SPIE Proceedings, New Methods in Microscopy and Low Light Imaging, 1161, 24-30, 1989). A typical approach in these intuitive approaches is to start with a first guess of the quantity to be reconstructed. The “forward projection” is then calculated from this first guess by applying the forward model, as in FIG. 3. The calculated projection is then compared with the actually measurement in some way. One way to compare is by subtracting the vector quantities. Another way is to take a ratio, as is done with the Gold's ratio method of image deconvolution (Sibarita, J. B., H. Magnin, and J. R. De Mey. 2002. Ultra-fast 4D microscopy and high throughput distributed deconvolution. In IEEE International Symposium on Biomedical Imaging, Washington, D.C. 669-772). The estimation is then modified based on this difference. For example, the correction calculated from the difference, may be spread evenly among all values being estimated.

It is possible to avoid collection of some or all of the calibration images and thereby simplify the experiment procedure. The ratio terms in Eq's. 6-12, which contain the M terms, may be treated as unknown vector elements that are estimated within the MLE procedure. This simplified approach will work for certain types of samples.

Stopping Criteria

As with all iterative algorithms, a quantitative stopping criterion is required to determine the number of iterations to execute. For brevity, three relatively simple stopping criteria will be described here, as examples. However, it is understood that any stopping criteria could be utilized. The first one, simply described, is to select a chosen number of iterations.

A second method involves an upperbound on the log-likelihood value. An upperbound is found by making the substitutions,

| | | |
|---|---|---|
| | μ_{DD}(x) = n_{DD}(x) , | (19) |
| | μ_{AA}(x) = n_{AA}(x) , | (20) |
| And | | |
| | μ_{DA}(x) = n_{DA}(x) . | (21) |

A lower bound is calculated by substituting the first guesses of the terms \hat{μ}_{DD}(x), \hat{μ}_{AA}(x), and \hat{μ}_{DA}(x) for μ_{DD}(x), μ_{AA}(x), and μ_{DA}(x) into Eq. 13. At the end of each iteration, then, the log-likelihood value of Eq. 13. When this value reaches a subset of the range between the calculated lower bound and upperbound, then the iterations are stopped. For example, we may stop the iterations after the log-likelihood reaches to within 0.01 of the top end of this range to the upperbound.

A third method is to stop the iterations if the change in log-likelihood from one iteration to the next is less than some specific small fraction of the current log-likelihood value, according to the expression:
|lₖ−lₖ₋₁|<w|lₖ₋₁|. (22)
Image Alignment

As noted above, FRET processing system 10 includes an image alignment system 12 for aligning images in the raw FRET set 22. Prior to carrying out any calculations, it is critical that all three of these images 22 represented by Eq's. 10, 11 and 12 are spatially aligned. It is common for the images to be misaligned by a few pixels because the filter wheel optical elements behave as refractors and they each refract the light differently. Misaligment may be due to other causes too, including stage vibration and motion of the sample due to live experiments. This alignment is critical. If misalignment occurs and is not corrected, there will be a significant F(x) erroneously calculated, and this may occur in regions of colocalized donor and acceptor even though there may not be FRET events occurring in those regions.

The invention utilizes an automated alignment algorithm 13 to align the images in the raw FRET image set before any calculation takes place. In one exemplary embodiment, alignment algorithm 13 uses a spatial cross correlation between the images to detect the amount of shift between images to a sub-pixel precision. The details of the method used for alignment are provide in the article: N. O'Connor, D. Bartsch, W. Freeman, A. Mueller, T. Holmes, Fluorescent infrared scanning-laser ophthalmoscope for three-dimensional visualization: automatic random-eye motion correction and deconvolution, Applied Optics, Vol. 37, No. 11, 2021-2033, 1998.

The alignment is affected for any two images iᵢ(x) and i₂(x) by executing the equation:
c(x)=α₁(x)*α₂(−x) (23)
where
α₁(x)=i₁(x)G(x) (24)
and
α₂(x)=i₂(x)G(x) (25)
and where G(x) represents a 2D Gaussian function of unit volume and chosen standard deviation. The term c(x) in Eq. 23 represents the cross-correlation of the two images a₁(x) and a₂(x) that are preconditioned by the window function G(x) according to Eq's. 24 and 25. The 2D shift value x′ by which iᵢ(x) is offset with respect to i₂(x) is found by locating the position of the maximum of c(x). The misalignment is corrected by shifting c(x) by −x′.

Obviously, other alignment techniques could be used without departing from the scope of the invention, including ones that correct for rotational misalignments.

FRET Efficiency

As also noted above, FRET processing system 10 also includes an efficiency calculation system 15. FRET efficiency E(x) is defined as the percentage of donor emissions that are quenched. From the FRET imaging algorithms this quantity is calculated according to:

$$\hat{E} ( x ) = \frac{\hat{F} ( x )}{{\hat{c}}_{d} ( x )} \qquad ( 26 )$$

where the hat denotes “estimate of.” From physical principles, it is theoretically expressed as:

$$E ( x ) = \frac{{R}_{0}^{6}}{{R}_{0}^{6} + {R}^{6}} \qquad ( 27 )$$

where R is the distance between the donor and acceptor molecule (usually a few nanometers) and Rₒ is the Forster distance and defined as the distance necessary to affect a 50% FRET efficiency. When considering quantum effects, this FRET efficiency E(x) represents the probability that any given donor deactivation event (i.e., the donor chromophore transfers from an excited state to a ground state) will be converted into a FRET event (i.e., quenched by transferring the energy nonradiatively to the acceptor chromophore) instead of being converted into a fluorescence emission photon. Rₒ a characteristic value that is documented by manufacturers for specific donor-acceptor pairs. E(x), Rₒ and Eq. 27 are important because they provide the underlying mathematical means for the experimental determination of the distance between the donor and acceptor molecules, which are bound to specific proteins, and thereby allows investigators to infer knowledge of the physical structure of these proteins.
Calibration of Filter Efficiencies

It should be noted that rather than estimating the excitation optical filter efficiencies S_{χoL} in the manner described above, these values could be obtained using a calibration approach. The efficiencies, as defined herein, are actually the integral of the multiplication of the following three terms: (1), the excitation light spectrum, (2), the dye's excitation (absorption) spectrum, and (3), the donor quantum yield. One could determine this integral analytically. The spectra of the dyes and the excitation light source (which could be a laser or an arc-lamp with an excitation filter) are published by the manufacturers, or they could be calibrated by the experimenter.

It is noted however that these measurements will not always be reliable for FRET experiments, because the spectra drift. The laser and arc-lamp spectra depend upon factors such as temperature, power-supply voltage and other factors. The spectra and quantum yield of the dyes depend upon their environment, which includes such factors as pH. Some dyes are more robust against such changes than others, but the safest assumption is that the spectra may drift. A similar argument holds for the emission filter efficiencies. The term S_{εoM}, theoretically, is equal to the integral of the dye's emission spectrum times the emission filter spectrum. More precisely, this term should be multiplied by the transmission spectra of all of the optical elements in the emission optics channel, including that of the photodector or CCD element. The safest assumption is that the dye's emission spectrum will change with environment, temperature, pH, humidity and other factors.

Calibration of Quantum Yield qₐ

Similarly, rather than attempting to estimate the specification of the quantum yield qₐ in the MLE in the manner described above, sometimes the values may be obtained from information provided by manufacturers. Even when these values are provided, they are not guaranteed because they are too dependent upon the environment into which the dye is being administered. The quantum yield, typically, will vary with pH and other chemical factor and such factors are difficult to control. Some dyes are more robust against these changes than others.

Experimental Results

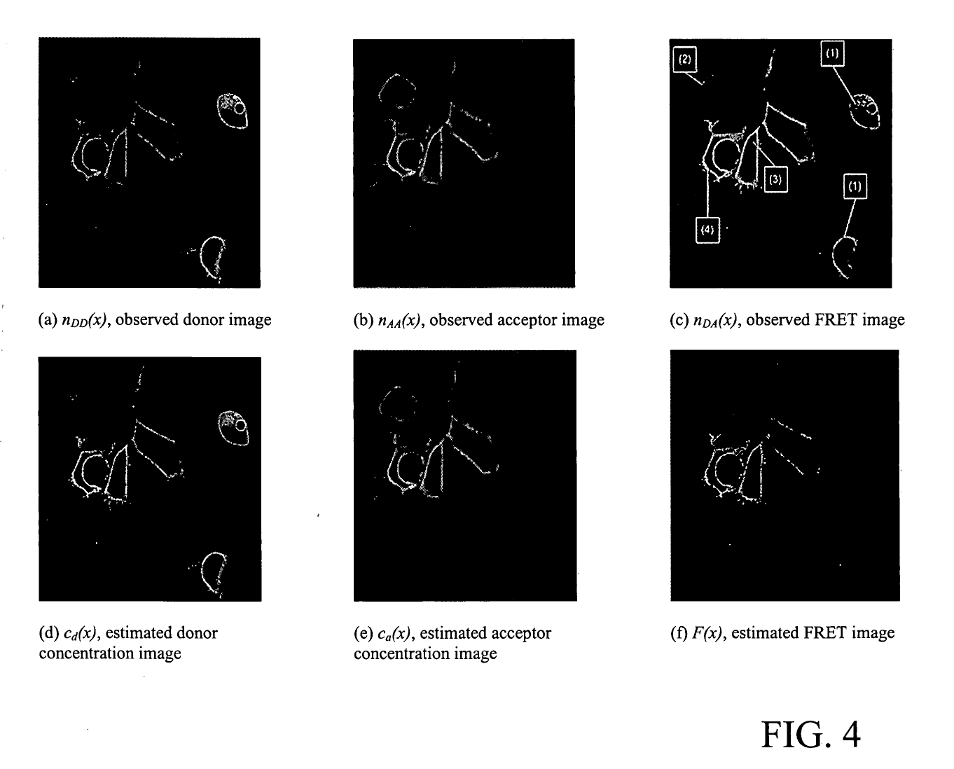

FIG. 4 shows a series of images that were processed in accordance with the techniques described herein. Table 2 describes the nine images utilized by the process.

TABLE 2

| | | | Excitation | Emission | |
|---|---|---|---|---|---|
| | | | filter tuned | filter tuned | |
| Image | Image | | to excite | to detect | |
| Groups | Notation | Sample: | which dye? | which dye? | Meaning |
| Calibration | μ_{dDD} | Donor only | Donor | Donor | The signal from a donor-only specimen |
| with donor | | | | | using the donor filter set |
| sample | μ_{dDA} | Donor only | Donor | Acceptor | The signal from a donor-only specimen |
| | | | | | using the FRET filter set |
| | μ_{dAA} | Donor only | Acceptor | Acceptor | The signal from a donor-only specimen |
| | | | | | using the Acceptor filter set |
| Calibration | μ_{aDD} | Acceptor | Donor | Donor | The signal from an acceptor-only |
| with | | only | | | specimen using the donor filter set |
| acceptor | μ_{aDA} | Acceptor | Donor | Acceptor | The signal from an acceptor-only |
| sample | | only | | | specimen using the FRET filter set |
| | μ_{aAA} | Acceptor | Acceptor | Acceptor | The signal from an acceptor-only |
| | | only | | | specimen using the acceptor filter set |
| raw FRET | n_{DD} | FRET(donor | Donor | Donor | The signal from an donor-and-acceptor |
| image set | | and acceptor) | | | specimen using the donor filter set |
| | n_{DA} | FRET(donor | Donor | Acceptor | The signal from an donor-and-acceptor |
| | | and acceptor) | | | specimen using the FRET filter set |
| | n_{AA} | FRET(donor | Acceptor | Acceptor | The signal from an donor-and-acceptor |
| | | and acceptor) | | | specimen using the acceptor filter set |

The three images that make up the raw FRET image set are n_{DD}(x), n_{AA}(x) and n_{DA}(x). They are called the observed donor image, the observed acceptor image and the observed FRET image, respectively.

This example illustrates the states that a pixel location can have with respect to these 3 images. In the observed FRET image (c), there are regions where only donor is present and bleed-through is apparent (1); only acceptor is present and cross-talk is apparent (2); there is colocalization of both donor and acceptor, but no FRET occurrence (3); and there is colocalization and FRET is occurring (4).

Condition 1 is apparent because in the observed donor image (a), the donor region is fluorescing, while in observed acceptor image (b), the same region has no acceptor fluorescence, and in observed FRET image (c), the donor is fluorescing, implying bleed-through.

Condition 2 is apparent because in observed acceptor image (b), an acceptor region is fluorescing, while in observed donor image (a), the same region shows no donor fluorescing, and in observed FRET image (c), the acceptor is fluorescing, implying cross-talk excitation of the acceptor.

In this example, four calibration images (μ_{dDD}, μ_{dDA}, μ_{aDA} and μ_{aAA}) are used, which are applied to Eq's. 5 through 9 to calculate the first guesses of the transfer efficiency terms S_{χaD} and S_{εdA}. Since the terms μ_{dAA} and μ_{aDD} are unavailable, they are set equal to zero in calculating Eq's. 6 and 9 for setting the first guesses of S_{χdA} and S_{εaD}.

The corrected donor concentration ĉ_{d}(x), corrected acceptor concentration ĉₐ(x) and corrected FRET image F(x) are shown in images (d)-(f). By comparing image (f) to (c), it is apparent that regions (1), (2) and (3) in the observed FRET image (c), which are caused by cross-talk and bleed-through, are properly removed in corrected FRET image (f). Region (4) in the observed FRET image (c), which represents a real FRET signal, is properly shown in the corrected FRET image (f).

FRET efficiencies of all four regions were calculated from the corrected images according to Eq. 26. Efficiencies of region (1), (2) and (3) were 2.48%, 1.71% and 4.53%, respectively; and the FRET efficiency of region (4) (i.e., averaged over the region) was 38.23%, while if using the uncorrected images, the FRET efficiencies was 62.571% for region (1), 236.91% for region (2), 149.95% for region (3) and 218.34% for region (4).

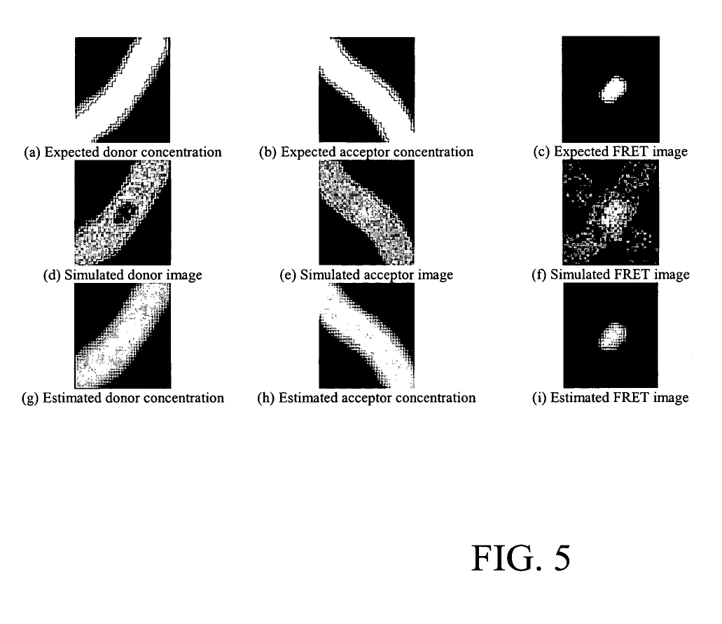

Synthetic images shown in FIG. 5 are used to analyze the MLE algorithm performance by comparing calculated results ĉ_{d}(x), ĉₐ(x) and \hat{F}(x) to the synthetic donor, acceptor and FRET concentrations ĉ_{d}(x), ĉₐ(x) and \hat{F}(x), respectively.

Three synthetic images (a)-(c) are generated with a computer program, and then they are applied to software that simulates the system shown in FIG. 3. The simulation software generates the six calibration images and three image of the raw FRET image set defined in Table 2. Poisson noise was added to all the simulated images. The signal-to-noise ratios (SNR's) for the observed donor image, observed acceptor image and observed FRET image were 7.58 dB, 7.98 dB, and 5.46 dB, respectively. The nine simulated images were processed with the MLE algorithm and the results are presented in images (g)-(i). The mean-square error was calculated in order to compare the estimated FRET efficiency F(x) to the true FRET efficiency F(x). This mean-square error was 3.398%, while the mean-square error of other common algorithms ranged from 5.032% to 12.024%.

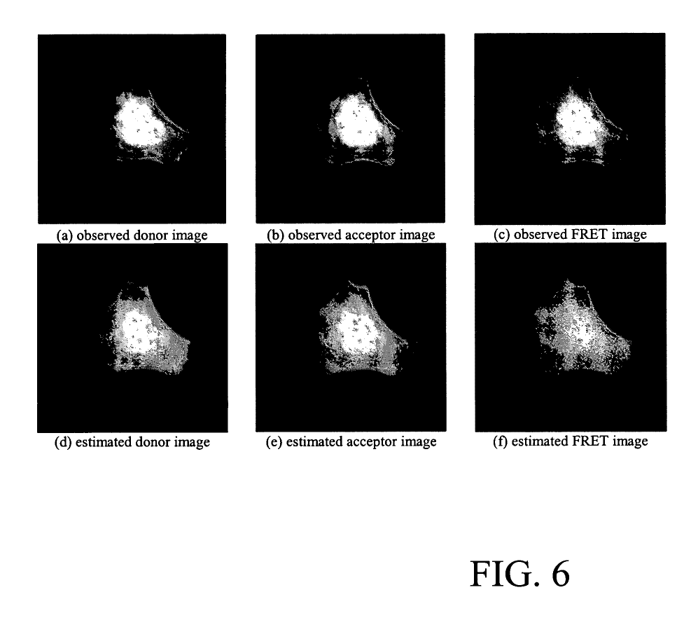

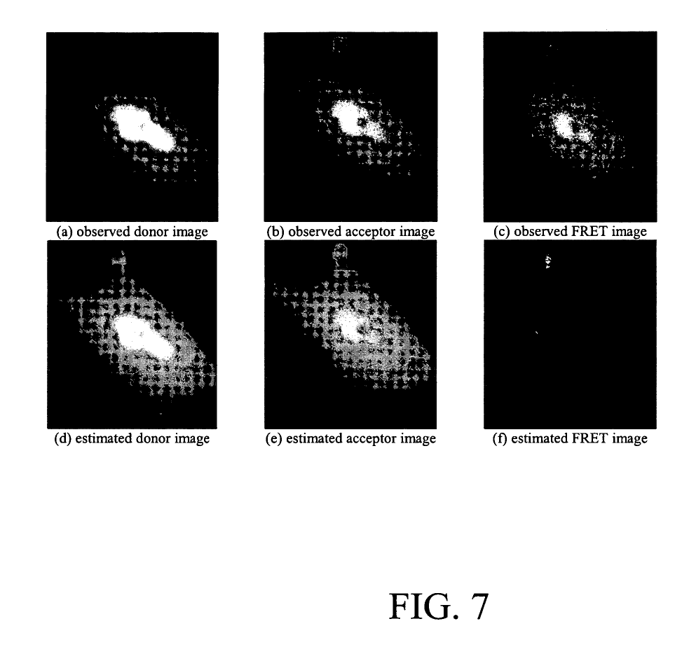

FIGS. 6 and 7 show the corrected images from a positive and negative control experiment, respectively. A positive control experiment uses a biological sample that is prepared under conditions where it is known that FRET will occur. Thus, there should be a clear signal in the corrected FRET image. As expected, a clear FRET signal is shown in FIG. 6, image (f). A negative control is an experiment in which a biological sample is prepared, with both donor and acceptor dyes, under conditions where it is known that FRET cannot occur. Thus, in principle one should expect no signal in the corrected FRET image.

In practice, we may expect a small signal level due to noise, residual misalignment and other causes, but this signal should be small. As expected, an image that is almost blank is shown in FIG. 7, image (f). The FRET efficiencies calculated from the MLE algorithm are 43.7% for the positive control and 0.65% for the negative control. In practice, the normal range of efficiencies for a positive control is considered to be higher than 5%. Therefore, the 43.7% and 0.65% numbers confirm the negative and positive controls.

It is understood that the systems, functions, mechanisms, methods, and modules described herein can be implemented in hardware, software, or a combination of hardware and software. They may be implemented by any type of computer system or other apparatus adapted for carrying out the methods described herein. A typical combination of hardware and software could be a general-purpose computer system with a computer program that, when loaded and executed, controls the computer system such that it carries out the methods described herein. Alternatively, a specific use computer, containing specialized hardware for carrying out one or more of the functional tasks of the invention could be utilized. The present invention can also be embedded in a computer program product, which comprises all the features enabling the implementation of the methods and functions described herein, and which—when loaded in a computer system—is able to carry out these methods and functions. Computer program, software program, program, program product, or software, in the present context mean any expression, in any language, code or notation, of a set of instructions intended to cause a system having an information processing capability to perform a particular function either directly or after either or both of the following: (a) conversion to another language, code or notation; and/or (b) reproduction in a different material form.

The foregoing description of the preferred embodiments of the invention has been presented for purposes of illustration and description. They are not intended to be exhaustive or to limit the invention to the precise form disclosed, and obviously many modifications and variations are possible in light of the above teachings. Such modifications and variations that are apparent to a person skilled in the art are intended to be included within the scope of this invention as defined by the accompanying claims.

1. A fluorescence resonance energy transfer (FRET) processing system, comprising:
an input for receiving image data, wherein the image data includes a raw FRET image set; and
an estimation image processing system that processes the image data and generates a set of estimation images, wherein the estimation image processing system includes an estimation algorithm and an iteration system.

2. The FRET processing system of claim 1, wherein the images of the raw FRET image set are of the same biological sample in which both a donor and an acceptor dye concentration is present, wherein a first image n_{DD}(x) is acquired with an excitation and an emission filter both tuned to the donor, and wherein a second image n_{DA}(x) is acquired with the excitation and emission filters tuned to the donor and acceptor, respectively, and wherein a third FRET image n_{AA}(x) is acquired with the excitation and emission filters both tuned to the acceptor.

3. The FRET processing system of claim 1, wherein the image data further comprises a set of calibration images.

4. The FRET processing system of claim 2, wherein the set of estimation images includes a corrected donor concentration image ĉ_{d}(x), a corrected acceptor concentration image ĉₐ(x) and a corrected FRET image \hat{F}(x).

5. The FRET processing system of claim 4, wherein the estimation algorithm utilizes a log-likelihood formula expressed as:
$$l = log \{ {\prod }_{x} [ P ( {μ}_{DD} ( x ) ; {n}_{DD} ( x ) ) ] [ P ( {μ}_{AA} ( x ) ; {n}_{AA} ( x ) ) ] [ P ( {μ}_{DA} ( x ) ; {n}_{DA} ( x ) ) ] \}$$
where the algorithm works by maximizing the this formula, P(•) represents a Poisson probability distribution, and its first and second arguments are its mean parameter and random variable, respectively, and are expressed as:
$$P ( {μ}_{LM} ( x ) ; {n}_{LM} ( x ) ) = \frac{{{μ}_{LM} ( x )}^{{n}_{LM} ( x )}}{{n}_{LM} ( x ) !} exp ( - {μ}_{LM} ( x ) )$$
$$wherein \text{:}$$
$$\begin{matrix}{μ}_{DD} ( x ) = [ {c}_{d} ( x ) - F ( x ) ] {S}_{χ dD} {S}_{ɛ d D} + {c}_{a} ( x ) \frac{{M}_{aDD}}{{M}_{aAA}} {S}_{χ a A} {S}_{ɛ a A} + \\ F ( x ) \frac{{M}_{aDD}}{{M}_{aDA}} {q}_{a} {S}_{χ d D} {S}_{ɛ a A} \\ {μ}_{DA} ( x ) = [ {c}_{d} ( x ) - F ( x ) ] \frac{{M}_{dDA}}{{M}_{dDD}} {S}_{χ dD} {S}_{ɛ d D} + {c}_{a} ( x ) \frac{{M}_{aDA}}{{M}_{aAA}} {S}_{χ a A} {S}_{ɛ a A} + \\ F ( x ) {q}_{a} {S}_{χ d D} {S}_{ɛ a A} \\ {μ}_{AA} ( x ) = [ {c}_{d} ( x ) - F ( x ) ] \frac{{M}_{dAA}}{{M}_{dDD}} {S}_{χ dD} {S}_{ɛ d D} + {c}_{a} ( x ) {S}_{χ a A} {S}_{ɛ a A} + \\ F ( x ) \frac{{M}_{dAA}}{{M}_{dDA}} {q}_{a} {S}_{χ d D} {S}_{ɛ a A}\end{matrix}$$
and wherein the subscripted S terms represent transfer efficiencies that encompass the optical filter's transfer efficiency, the subscripted M terms represent a summation of the image taken with a calibration sample, and the subscripted n terms represent the raw unprocessed image data collected from the microscope and camera system.

6. The FRET system of claim 1, further comprising an image alignment system that automatically aligns the inputted images of the raw FRET image set.

7. The FRET system of claim 6, wherein the image alignment system utilizes a cross-correlation algorithm.

8. The FRET system of claim 1, further comprising a system that calculates a FRET efficiency.

9. The FRET system of claim 1, wherein the estimation algorithm is selected from the group consisting of: maximum a posteriori, least squares, minimum mean-square-error, maximum entropy, maximum cross-entropy and maximum likelihood estimation.

10. The FRET system of claim 1, wherein the iteration system uses a technique selected from the group consisting of: expectation-maximization, steepest ascent, conjugate gradient, simulated annealing and linear programming.

11. A fluorescence resonance energy transfer (FRET) processing system, comprising:
an input system for receiving image data, wherein the image data includes a raw FRET image set;
an image alignment system that automatically aligns pixels in at least two images of the raw FRET image set; and
an image processing system that processes the raw FRET image set and generates a corrected FRET image F(x).

12. The FRET processing system of claim 11, wherein the images in the raw FRET image set are of the same biological sample in which both a donor and an acceptor dye concentration are present, wherein a first image n_{DD}(x) is acquired with an excitation and an emission filter both tuned to the donor, and wherein a second image n_{AA}(x) is acquired with the excitation and emission filters both tuned to the acceptor, and wherein a third image n_{DA}(x) is acquired with the excitation and emission filters both tuned to the donor and acceptor, respectively.

13. The FRET processing system of claim 11, wherein the image alignment system utilizes a cross-correlation algorithm.

14. The FRET processing system of claim 11, wherein the image processing system utilizes an estimation algorithm to calculate pixel values for a corrected FRET image \hat{F}(x).

15. The FRET processing system of claim 11, wherein the image set further comprises a calibration image set.

16. A method for processing fluorescence resonance energy transfer FRET image (FRET) data, comprising:
providing a raw FRET image set for a sample; and
generating a set of estimation images with an estimation algorithm that uses image data from the raw FRET image set.

17. The method of claim 16, comprising the further steps of providing a set of calibration images nad using the calibration images to generate the set of estimation images.

18. The method of claim 17, wherein the raw FRET image set and the calibration image set are obtained with a device selected from the group consisting of a confocal microscope and a widefield microscope.

19. The method of claim 16, wherein the raw FRET image set comprises a first image n_{DD}(x) acquired with an excitation and an emission filter both tuned to a donor, a second image μ_{AA}(x) acquired with the excitation and emission filters both tuned to the acceptor, and a third image μ_{DA}(x) acquired with the excitation and emission filters tuned to the donor and acceptor, respectively.

20. The method of claim 19, wherein the estimation images comprise a corrected donor concentration image ĉ_{d}(x), a corrected acceptor concentration image ĉₐ(x) and a corrected FRET image \hat{F}(x).

21. The method of claim 20, wherein the estimation algorithm utilizes a log-likelihood formula expressed as:
$$l = log \{ {\prod }_{x} [ P ( {μ}_{DD} ( x ) ; {n}_{DD} ( x ) ) ] [ P ( {μ}_{AA} ( x ) ; {n}_{AA} ( x ) ) ] [ P ( {μ}_{DA} ( x ) ; {n}_{DA} ( x ) ) ] \}$$
where the algorithm works by maximizing the this formula, P(•) represents a Poisson probability distribution, and its first and second arguments are its mean parameter and random variable, respectively, and are expressed as:
$$P ( {μ}_{LM} ( x ) ; {n}_{LM} ( x ) ) = \frac{{{μ}_{LM} ( x )}^{{n}_{LM} ( x )}}{{n}_{LM} ( x ) !} exp ( - {μ}_{LM} ( x ) )$$
$$wherein \text{:}$$
$$\begin{matrix}{μ}_{DD} ( x ) = [ {c}_{d} ( x ) - F ( x ) ] {S}_{χ dD} {S}_{ɛ d D} + {c}_{a} ( x ) \frac{{M}_{aDD}}{{M}_{aAA}} {S}_{χ a A} {S}_{ɛ a A} + \\ F ( x ) \frac{{M}_{aDD}}{{M}_{aDA}} {q}_{a} {S}_{χ d D} {S}_{ɛ a A} \\ {μ}_{DA} ( x ) = [ {c}_{d} ( x ) - F ( x ) ] \frac{{M}_{dDA}}{{M}_{dDD}} {S}_{χ dD} {S}_{ɛ d D} + {c}_{a} ( x ) \frac{{M}_{aDA}}{{M}_{aAA}} {S}_{χ a A} {S}_{ɛ a A} + \\ F ( x ) {q}_{a} {S}_{χ d D} {S}_{ɛ a A} \\ {μ}_{AA} ( x ) = [ {c}_{d} ( x ) - F ( x ) ] \frac{{M}_{dAA}}{{M}_{dDD}} {S}_{χ dD} {S}_{ɛ d D} + {c}_{a} ( x ) {S}_{χ a A} {S}_{ɛ a A} + \\ F ( x ) \frac{{M}_{dAA}}{{M}_{dDA}} {q}_{a} {S}_{χ d D} {S}_{ɛ a A}\end{matrix}$$
and wherein the subscripted S terms represent transfer efficiencies that encompass the optical filter's transfer efficiency, the subscripted M terms represent a summation of the image taken with a calibration sample, and the subscripted n terms represent the raw unprocessed image data collected from the microscope and camera system.

22. The method of claim 16, wherein the step of providing the raw FRET image set includes the step of aligning the images.

23. An image processing system, comprising:
an image collection system for collecting image data including a raw FRET image set; and
a FRET processing system for processing the image data and generating a set of estimation images using an estimation algorithm.

24. The image processing system of claim 23,
wherein the raw FRET image set includes a first image acquired with an excitation and an emission filter both tuned to a donor, a second image acquired with the excitation and emission filters tuned to the donor and an acceptor, respectively, and a third image acquired with the excitation and emission filters both tuned to the acceptor; and
wherein the estimation images comprise a corrected donor concentration image, a corrected acceptor concentration image and a corrected FRET image.

25. The image processing system of claim 23, wherein the image data includes a set of calibration images.
